(19) United States
(12) Patent Application Publication
(10) Pub. No.: US 2017/0290981 A1
(43) Pub. Date: Oct. 12, 2017

(54) Drug Delivery Device with Piston Driver Distal Feature

(71) Applicant: Novo Nordisk A/S, Bagsvaerd (DK)

(72) Inventors: Jesper HOEHOLT, Melby (DK); Jens Aage MUNK, Oelstykke (DK)

(73) Assignee: Novo Nordisk A/S, Bagsvaerd (DK)

(21) Appl. No.: 15/511,932

(22) PCT Filed: Sep. 17, 2015
(86) PCT No.: PCT/EP2015/071321

§ 371 Date: Mar. 16, 2017

(30) Foreign Application Priority Data
Sep. 18, 2014 (EP) 14185312.7

(51) Int. Cl. A61M 5/20; A61M 5/315

(52) U.S. Cl. CPC A61M 5/20; A61M 5/315; A61M 5/31511; A61M 2005/31508; A61M 5/172

(57) ABSTRACT

Drug delivery device adapted to receive a cartridge, comprising drug expelling means with a piston driver comprising a distal end adapted to abut and axially move the piston of a loaded cartridge to thereby expel an amount of drug from the cartridge, and a motor assembly for moving the piston driver. The piston driver distal end comprises flexible centring means adapted to frictionally engage a cartridge inner surface proximally of the piston, thereby centring the cartridge and the piston driver distal end relative to the each other.

The present invention generally relates to a motorized drug delivery device adapted to receive a drug filled cartridge and subsequently expel a dose therefrom.

### BACKGROUND OF THE INVENTION

In the disclosure of the present invention reference is mostly made to the treatment of diabetes by subcutaneous drug delivery, either discrete or continuous, however, this is only an exemplary use of the present invention.

The most common type of durable drug delivery devices adapted to receive a drug filled cartridge and expel a discrete dose of a desired size therefrom are driven by manual means or by a spring energized during dose setting, the cartridge being of the type comprising an axially displaceable piston having an initial proximal position and which is moved distally by a piston rod. Subcutaneous drug delivery takes place via an injection needle arranged in fluid communication with the cartridge. The device may be pen-formed or in the form of a more box-shaped so-called doser. In order to improve convenience, user-friendliness and provide additional features, e.g. detection and storing of expelling data, drug delivery devices have been provided with electrically driven means, typically in the form of an electronically controlled motor driving a piston rod through a gear arrangement, e.g. as shown in U.S. Pat. No. 6,514,230 and US 2011/306927.

Whereas motorized drug delivery devices for treatment of diabetes by discrete injections of e.g. insulin are used relatively rarely, in the field of continuous drug delivery motorized drug delivery devises have been used widely for decades. The latter type of devices are generally known as infusion pumps and are normally engineered to very high standards and are correspondingly very expensive.

Although a motorized drug delivery device for discrete injections of drug also has to meet very high safety standards, the cost issue is more important as the relatively inexpensive mechanical drug delivery devices, e.g. of the pen-type, to most users are an acceptable alternative. Correspondingly, to make the higher expense acceptable to the user additional advantages should be offered. To reduce the risk of free flow WO 2007/118907 discloses a drug-filled cartridge in which the piston is provided with a friction enhancing member.

Having regard to the above, it is an object of the present invention to provide a motorized drug delivery device which is reliable and provides a high degree of user-friendliness in a cost-effective way.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in accordance with a first aspect of the invention a drug delivery device is provided comprising a compartment adapted to receive and hold a drug-filled cartridge, the cartridge comprising an outlet, a generally cylindrical proximal portion and an axially displaceable piston in sliding engagement with the cartridge inner surface. The device further comprises drug expelling means comprising a piston driver comprising a distal end adapted to abut and axially move, directly or indirectly, the piston of a loaded cartridge to thereby expel an amount of drug from the cartridge through the outlet. The piston driver distal end comprises flexible centring means adapted to frictionally engage a cartridge inner surface proximally of the piston, thereby centring the cartridge and the piston driver distal end relative to the each other. By the term abut is indicated that the piston driver is not adapted to be coupled to the piston, e.g. by a threaded connection, but is allowed to be axially withdrawn from the piston.

By this arrangement the flexible centring means can be designed to add an axial resistance higher than the force needed to make sure that all mechanical play is removed in the system. This will make sure that the piston rod is not moving before the play is removed from the system. The flexible centring means will also avoid that the weight of the piston rod and motor can cause the plunger to move unintended. Further, the flexible centring means will also help assure that the piston driver stays centred when lateral forces are applied, e.g. in case the drug delivery device is dropped on a hard surface, this preventing a large-diameter piston rod to come into contact with the cartridge which may damage the latter, especially in case the piston rod is manufactured from metal.

In an exemplary embodiment the piston driver distal end comprises sensor means, e.g. in the form of a force sensor or a proximity sensor. In a motorized drug delivery device such a sensor can be used to detect when the piston has reached the cartridge piston. In a standard cartridge the piston distal surface is often provided with a number of ribs or protrusions which may result in differing measurements depending on the location of the sensor means relative to the ribs. As the tolerance on the internal diameter in a standard glass cartridge is relatively large the placement tolerance on the ribs on the cartridge piston may be correspondingly large. These tolerances will cause a possible misalignment between the sensor means and the piston ribs. If the sensor has contact with the ribs on the plunger it might disturb the signal and influence the precision in the system. The centring feature will provide more uniform measurements, e.g. by designing a piston driver distal contact interface which will not engage the ribs.

In an exemplary embodiment the compartment comprises a distal opening through which a cartridge can be inserted and subsequently removed, the arrangement providing a frontloaded drug delivery device. The drug delivery device may further comprise closure means operatable between an open state allowing a cartridge to be inserted or removed, and a closed state in which a received cartridge is held in an operational mounted position. In such an arrangement the friction between the flexible centring means and the cartridge can be used to move the cartridge out of the device.

More specifically, in an exemplary embodiment a controller is provided which is adapted to detect a cartridge exchange state when the closure means is operated from the closed to the open state with a loaded cartridge. When a cartridge exchange state is detected the controller operates the motor assembly to (i) move the piston driver in the proximal direction, the flexible centring means thereby sliding on the cartridge inner surface in frictional engagement, and (ii) move the piston driver in the distal direction, the flexible centring means thereby moving the cartridge distally through the compartment distal opening, thereby allowing a user to grip the cartridge distal portion. The piston driver can then be fully retracted and the user can insert a new cartridge in the device without applying a force to overcome the friction between the cartridge and piston driver centring means.

The above-described drug delivery devices may be provided in combination with a drug-filled cartridge, the cartridge comprising an outlet, a generally cylindrical proximal portion and an axially displaceable piston in sliding engagement with the cartridge inner surface, the cartridge comprising an inner surface proximally of the piston.

As used herein, the term “drug” is meant to encompass any flowable medicine formulation capable of being passed through a delivery means such as a cannula or hollow needle in a controlled manner, such as a liquid, solution, gel or fine suspension, and containing one or more drug agents. Representative drugs include pharmaceuticals such as peptides (e.g. insulins, insulin containing drugs, GLP-1 containing drugs as well as derivatives thereof), proteins, and hormones, biologically derived or active agents, hormonal and gene based agents, nutritional formulas and other substances in both solid (dispensed) or liquid form. In the description of the exemplary embodiments reference will be made to the use of insulin containing drugs, this including analogues thereof as well as combinations with one or more other drugs.

### BRIEF DESCRIPTION OF THE DRAWINGS

In the following exemplary embodiments of the invention will be further described with reference to the drawings, wherein

FIG. 1 shows schematically an embodiment of a drug delivery device,

FIGS. 2 and 3 show in greater detail an embodiment of a drug delivery device platform,

FIG. 4 shows schematically an embodiment of a base member with centring means, and

FIG. 5 shows schematically an embodiment of a piston head with centring means.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The term “assembly” does not imply that the described components necessarily can be assembled to provide a unitary or functional assembly during a given assembly procedure but is merely used to describe components grouped together as being functionally more closely related.

Before turning to a detailed description of an exemplary embodiment of the invention a schematic representation of drug delivery device with a telescopic motor-in-piston drive assembly will be described to better provide an understanding of the general working principle of such an arrangement.

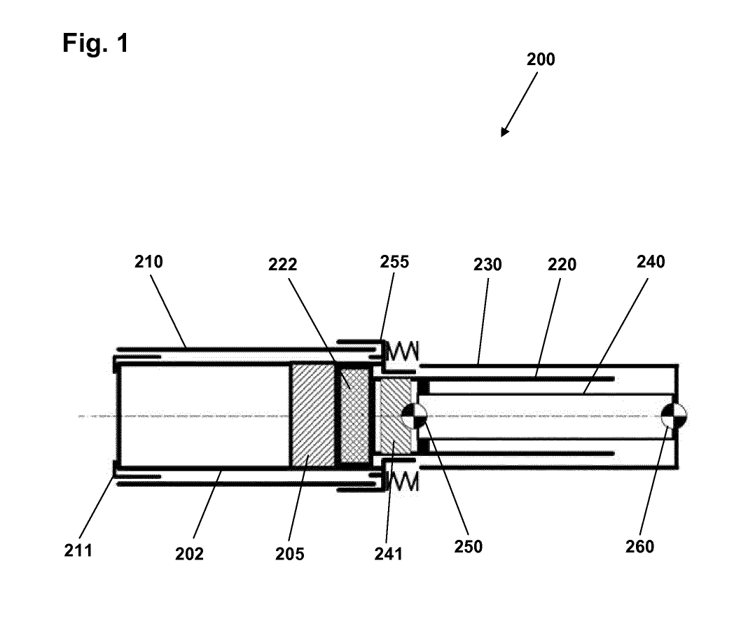

More specifically, FIG. 1 shows schematically an exemplary drug delivery device 200 comprising a front-loaded compartment portion 210 adapted to receive and hold a drug-filled generally cylindrical cartridge 202 by means of distally arranged cartridge holding means 211, a main portion 230 in which a telescopic motor-in-piston drive assembly 220, 240 is arranged, and a bias assembly 255 providing a biasing distally directed force on a loaded cartridge. Although shown as two components, the compartment portion and the main portion are rigidly connected to each other or formed as a single component providing a chassis or platform for other components of the device.

The drive assembly comprises an outer piston drive tube 220 with an inner thread, a motorgear assembly 240 having a distal portion and a proximal portion, the distal portion comprising a rotatable drive shaft defining a z-axis and to which is mounted a drive member 241 comprising an outer thread in engagement with the piston drive tube inner thread. The motorgear assembly is arranged axially non-displaceable and non-rotational relative to the chassis, the drive member is mounted axially non-displaceable and non-rotational on the drive shaft, and the piston drive tube is arranged axially displaceable but non-rotational relative to the chassis and thus also to the motor assembly, whereby rotation of the drive shaft results in axial, non-rotational displacement of the piston drive tube relative to the chassis, the piston drive tube being adapted to abut and axially move distally, directly or indirectly, the piston of a loaded cartridge to thereby expel drug from the cartridge. In the shown embodiment the piston drive tube is provided with a distal drive head 222 adapted to abut the piston 205 of a loaded cartridge 202.

The shown embodiment of FIG. 1 is provided with optional kinematic joints to allow additional degrees of freedom between components. More specifically, the drive shaft is connected to the drive member via a first flexible joint 250, and the motor-gear assembly proximal portion is connected to the chassis via a second flexible joint 260, the flexible joints being designed to provide a rotational lock between the connected components, yet allowing the components to bend or flex relative to each other.

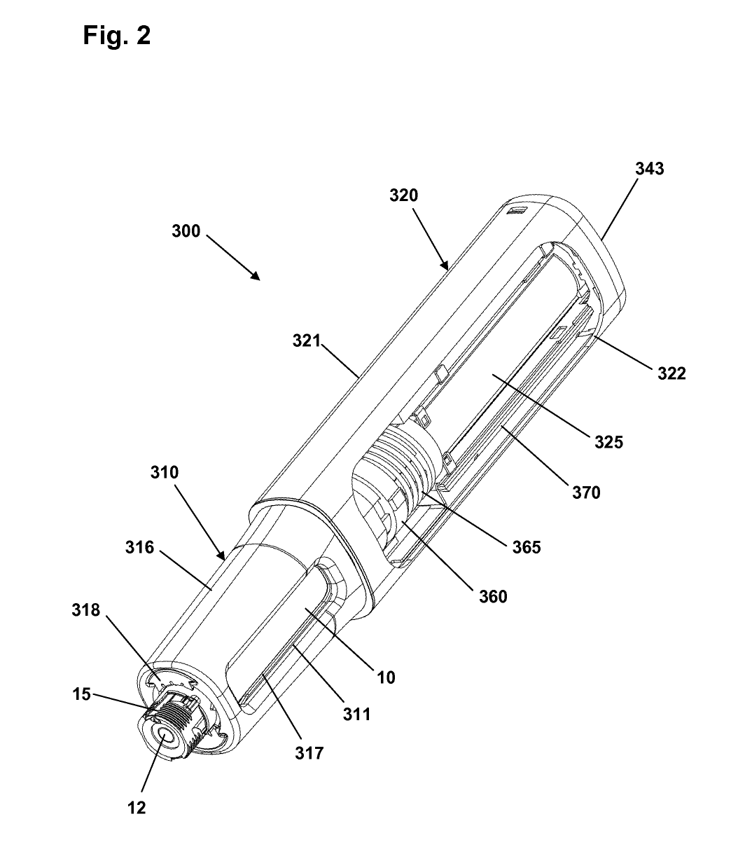

Turning to FIG. 2 a motor-in-piston drug delivery device 300, suitable as a platform for embodiments of the present invention, will be described. More specifically, the device comprises a cap part (not shown) and a main part having a proximal body or drive assembly portion 320 with a housing 321 in which a drug expelling mechanism and associated electronics 370 are arranged, and a distal cartridge holder assembly 310 forming a compartment in which a drug-filled transparent cartridge 10 can be arranged and retained in place, the cartridge holder assembly comprising a pair of opposed inspection openings 311. The housing comprises an opening 322 adapted to receive a display frame member (not shown) in which a LCD as well as user input keys are mounted. With the frame member removed, it can be seen that the device comprises a generally tubular chassis member 325, in which a generally cylindrical expelling assembly is mounted (see below). The device further comprises a control assembly 370, a bias assembly comprising a bias member 360 and a spring 365, and a proximal release button 343. A pair of dose setting input keys (not shown) serves to manually set a desired dose of drug shown in the LCD and which can then be expelled when the release button 343 is actuated. The device is designed to be loaded by the user with a new cartridge through a distal receiving opening in the cartridge holder assembly.

The cartridge 10 comprises a cylindrical body portion, a distal outlet portion 12 with a distal needle-penetrable septum, and an axially displaceable piston having a proximal surface allowing a piston driver forming part of the expelling mechanism (see below) to abut the piston. The cartridge may for example contain an insulin, a GLP-1 or a growth hormone formulation. The cartridge is provided with distal coupling means in the form of a needle hub mount 15 having, in the shown example, combined thread and bayonet coupling means, each being adapted to engage an inner thread or bayonet coupling means of a corresponding hub of a needle assembly. The shown exemplary hub mount further comprises a circumferential flange with a number of distally facing pointed projections serving as a coupling means for the cartridge holder assembly as will be described in more detail below. A hub mount of the shown type is described in U.S. Pat. No. 5,693,027. Alternatively the needle hub mount may be formed as part of the cartridge holder, e.g. in the form of a “split” hub mount having two parts arranged on each side of the gripping shoulders.

As shown, the cartridge holder assembly 310 has the same general appearance as a traditional cartridge holder which is detachably coupled to the housing by e.g. a threaded coupling or a bayonet coupling and into which a new cartridge can be received as well as removed through a proximal opening, i.e. it comprises no additional user operated release or locking means. Instead, what appears merely to be the cartridge holder per se is in fact user operated coupling means in the form of an outer rotatable tubular actuation sleeve 316 operated by the user to control movement of cartridge holding means in the form of an inner cartridge holder member 317 to thereby open and close gripping shoulders 318 configured to grip and hold a cartridge. More specifically, each gripping shoulder is provided with a plurality of gripping teeth spaced circumferentially to provide a plurality of gaps, each tooth having a triangular configuration with a proximally oriented pointed end, thereby creating a plurality of gaps having a distally oriented pointed configuration, this allowing the above-described distally facing pointed projections on the cartridge to be received between the teeth to thereby serve as a gripping means when the cartridge holding means has been moved into engagement with the cartridge. In this way an easy-to-use front loaded drug delivery device is provided which appears as a traditional rear loaded device and which is also actuated by rotational movement to mount and remove a cartridge, the resemblance providing for ease of acceptance and adaptation among users accustomed to traditional types of rear loaded drug delivery devices.

When it is time to mount a new cartridge the outer tube member 316 is rotated e.g. 90 degrees by which action the gripping shoulders 318 are moved distally and slightly outwards, this allowing the mounted cartridge to be removed. For ease of operation the cartridge may be moved distally a certain distance as the shoulders are moved, e.g. by engagement with arms forming the gripping shoulders and/or by additional spring means providing a biasing distally directed force (see below). Depending on the design of the locking and actuation mechanism the gripping shoulders may be able to be left in the open position or they may be retracted automatically as the outer tube member is rotated backwards by return spring means. Whether or not a spring is provided the cartridge holder may be provided with locking means allowing the outer tube member to be securely parked in either the open or closed position, e.g. by a rotational snap lock. When a new cartridge is inserted the drive expelling means has to be in a state allowing a new cartridge with a proximally positioned piston to be inserted. An exemplary embodiment providing this functionality will be described below.

Turning to FIG. 3 a cross-sectional view of the drug delivery device 300 of FIG. 2 is shown with a mounted cartridge 10 and with the piston tube 330 (see below) in a fully retracted position. More specifically, the actuation sleeve 316 has been rotated to its operational position and the cartridge holder gripping shoulders 318 have been retracted to their closed position thereby retracting the cartridge to its fully inserted position, thereby also moving the bias member 360 proximally against the bias of the spring 365. In the shown embodiment a cartridge switch 375 is hereby being actuated, this providing a signal to the device controller that two actions can be assumed to have taken place: (i) a cartridge has been inserted and (ii) the cartridge holder has been closed, this initiating that the drive head is moved distally into contact with the cartridge piston. In the shown embodiment it is contemplated that detection of contact between the drive head and the piston is detected by electronic sensor means arranged in the drive head, e.g. using force sensing or proximity detection as disclosed in WO 2013/144152.

FIG. 3 also shows the expelling assembly in greater detail. More specifically, the expelling assembly is in the form of a motor-in-piston assembly comprising an interior motor and gearbox drive assembly mounted axially and rotationally locked to the proximal end of the chassis, and an outer axially displaceable piston tube 330 with a distal drive head 332 adapted to abut the piston 11 of a loaded cartridge, the piston tube comprising a number of guide projections adapted to non-rotationally engage corresponding guide means of the chassis.

The motor-gear drive assembly comprises a tubular main portion composed of a proximal motor assembly 351 and a distal gearbox assembly 352 having a rotatable drive shaft 353 defining a z-axis of rotation. The assembly further comprises a distal cylindrical drive member 355 having an outer thread adapted to be arranged in engagement with the piston drive tube inner thread. At the proximal end a disc-formed chassis connector 356 is arranged. In the shown embodiment the drive assembly is provided with flexible joints in the form of a distal universal joint 357 arranged between the drive shaft and the drive member and a proximal universal joint 358 arranged between the motor assembly proximal portion and the chassis tube proximal portion. A corresponding drive assembly is described in greater detail in patent application EP 14166859.0, which is hereby incorporated by reference.

A number of further details can be seen in FIG. 3. The release button 343 is received in the housings proximal opening with a spring providing a proximally directed biasing force on the button. A flexible ribbon 376 with a plurality of conductors is arranged with a U-bend between the electronics portion 370 and the sensors (not shown) arranged in the piston head, this allowing the piston tube and piston head to travel axially with the U-bend moving correspondingly.

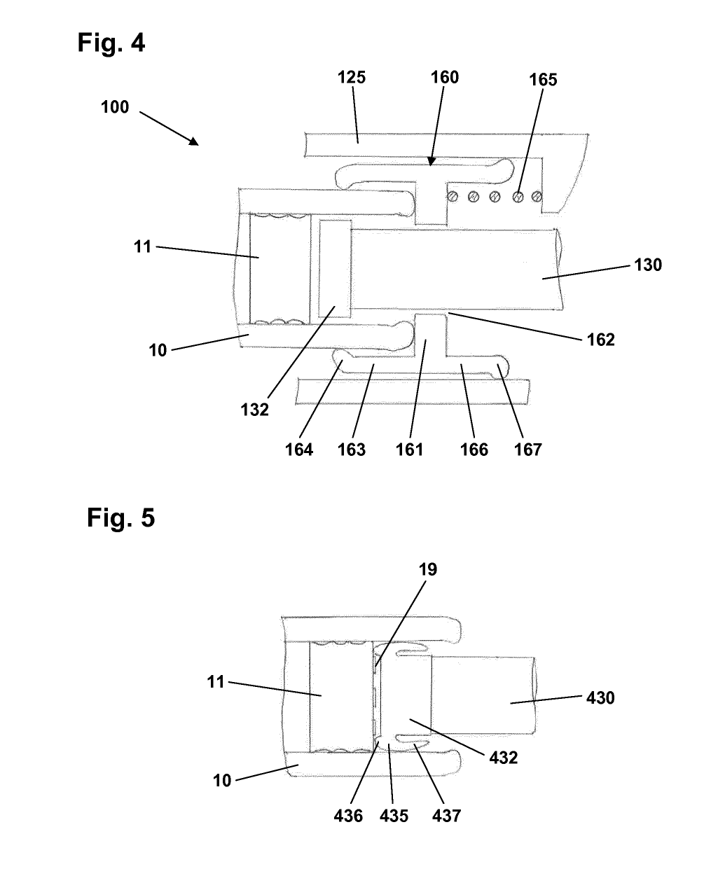

Turning to FIG. 5 an embodiment of the present invention will be described. More specifically, FIG. 5 discloses a piston driver in the form of piston tube 430 having a distal head 432 with flexible centring means in the form of a number (here: four) circumferentially arranged flexible arms 435 adapted to laterally engage a cartridge 10 inner surface proximally of the piston 11. Each arm comprises a distal end 436 adapted to engage the piston proximal surface peripherally and thus free of the piston ribs 19, and a proximally extending free portion 437 adapted to frictionally engage the cartridge inner surface.

By this arrangement the flexible centring means can be designed to add an axial resistance higher than the force needed to make sure that all mechanical play is removed in the system. This will make sure that the piston rod is not moving before the play is removed from the system. Further, the centring feature will provide more uniform measurements when sensor means is provided in the piston driver.

With reference to the embodiment of FIG. 3 it was described that during cartridge change the cartridge may be moved out of the device distal opening a certain distance as the shoulders are moved distally, e.g. by engagement with arms forming the gripping shoulders and/or by additional spring means providing a biasing distally directed force. For this functionally to be preserved when the piston driver distal end is provided with the frictional centring means of FIG. 5 the force moving the cartridge distally would have to overcome the friction between the piston head and the cartridge.

Alternatively the controller may be adapted to detect a cartridge exchange state when the holding means is operated from the closed to the open state with a loaded cartridge. When a cartridge exchange open state is detected the controller operates the motor assembly to (i) move the piston driver in the proximal direction, the flexible centring means thereby sliding on the cartridge inner surface in frictional engagement, and (ii) move the piston driver in the distal direction, the flexible centring means thereby moving the cartridge distally through the compartment distal opening, thereby allowing a user to grip the cartridge distal portion. The piston driver can then be fully retracted and the user can insert a new cartridge in the device without applying a force to overcome the friction between the cartridge and piston driver centring means.

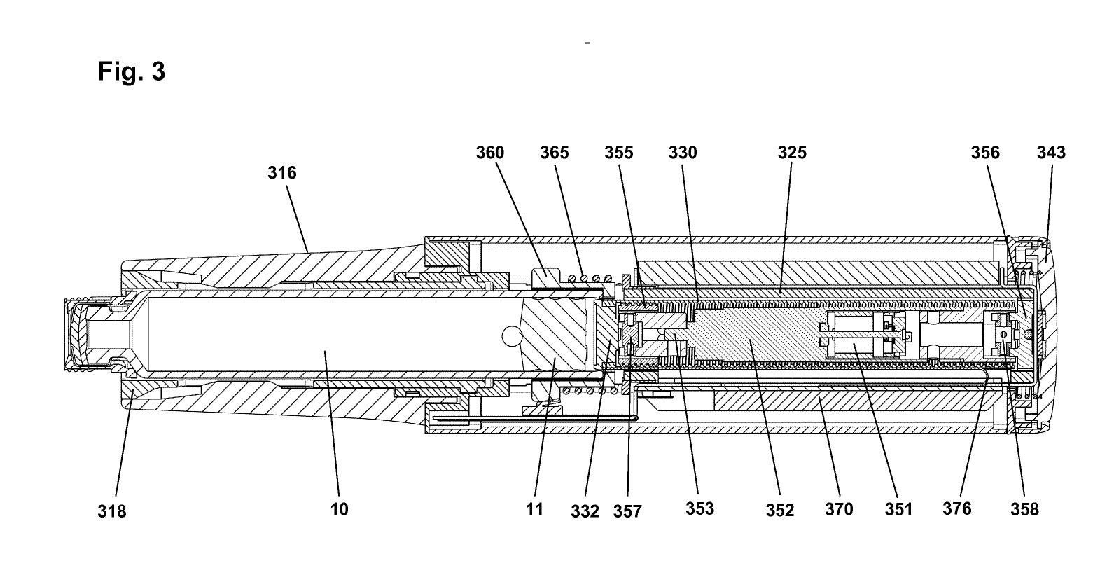

Turning to FIG. 4 a further issue for a drug delivery device of the type shown in FIG. 3 will be described. As appears from the above description of an exemplary motor-in-piston drug delivery device, the limiting dimension for the piston and thus for the drive mechanism per se is the interior diameter of the cartridge. A further issue is the fact that conventional drug cartridges for e.g. insulin and growth hormone are manufactured from glass for which reason it should be avoided that the piston, especially when manufactured from metal, can come into contact with the glass cartridge, e.g. after a drop to the floor, this requiring a small piston diameter.

As all components are manufactured with a given tolerance the actual dimension selected for the outer piston tube diameter has to take into account the tolerances for all relevant components. Thus, the smaller the aggregate tolerances between the piston tube outer circumference and the cartridge inner circumference the larger the outer diameter for the piston tube can be selected.

Addressing this issue a base member is provided, the base member having (i) a central opening guiding and centring the piston, and (ii) flexible portions engaging the cartridge outer circumference and centring it relative to the piston, this reducing the tolerance chain between piston and cartridge making a larger diameter piston possible. The base member may be provided with additional flexible portions centring the base member relative to the chassis.

When the base member is centring the cartridge, the tolerance chain from the cartridge to the piston tube will be as short as possible because they are aligned in the same component. When the base member is centred in relation to the chassis, the tolerance chain from the piston diameter to the chassis will also be as short as possible. Each of the centring features can be implemented independently if only one is required.

FIG. 4 shows in a schematic partial representation a motor-in-piston drug delivery device 100 with a base member in the form of a bias member 160, the drug delivery device and the bias member being of the same general design as in the FIG. 3 embodiment, i.e. comprising an axially displaceable piston tube 130 with a distal drive head 132 adapted to abut and engage the piston 11 of a loaded cartridge 10, as well as a bias member 160 axially moveable between a distal position when no cartridge is mounted and a proximal position when a cartridge is mounted and engaged by the bias member, the bias member being biased towards its distal position by a helical spring 165 arranged between the device chassis 125 and the bias member. The bias member 160 comprises a main portion 161 with a central passage 162 in which a portion of the piston driver is arranged axially displaceable. The bias member further comprises first flexible centring means in the form of a number (here: four) distally extending flexible arms 163 with inwardly directed distal ends 164 adapted to receive and frictionally engage the proximal outer circumference of a cartridge and centre it relative to the central opening and thereby to the piston driver. The bias member further comprises second flexible centring means in the form of a number (here: four) proximally extending flexible arms 166 with outwardly directed proximal ends 167 adapted to slidingly engage the device chassis 125 and centre the bias member relative to the housing.

The piston tube distal centring means may be provided in combination with the above-described bias member 160 of FIG. 4.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A drug delivery device comprising:
a compartment adapted to receive and hold a drug-filled cartridge, the cartridge comprising an outlet, a generally cylindrical proximal portion and an axially displaceable piston in sliding engagement with the cartridge inner surface,
drug expelling structure comprising:
a piston driver comprising a distal end adapted to abut and axially move, directly or indirectly, the piston of a loaded cartridge to thereby expel an amount of drug from the cartridge through the outlet, and
a motor assembly for moving the piston driver,
wherein the piston driver distal end comprises flexible centering structure adapted to frictionally engage a cartridge inner surface proximally of the piston, thereby centring the cartridge and the piston driver distal end relative to the each other.

2. A drug delivery device as in claim 1, wherein the flexible centering structure comprises a number of proximally extending flexible arms.

3. A drug delivery device as in claim 1, wherein the piston driver distal end comprises sensor structure.

4. A drug delivery device as in claim 3, wherein the sensor structure is either a force sensor or a proximity sensor.

5. A drug delivery device as in claim 1, wherein the compartment comprises a distal opening through which a cartridge can be inserted and subsequently removed, the arrangement providing a frontloaded drug delivery device.

6. A drug delivery device as in claim 5, further comprising closure structure operable between an open state allowing a cartridge to be inserted or removed, and a closed state in which a received cartridge is held in an operational mounted position.

7. A drug delivery device as in claim 6, further comprising a controller adapted to:
detect a cartridge exchange state when the closure structure is operated from the closed to the open state with a loaded cartridge,
when a cartridge exchange state is detected operate the motor assembly to:
move the piston driver in the proximal direction, the flexible centering structure thereby sliding on the cartridge inner surface in frictional engagement, and
move the piston driver in the distal direction, the flexible centering structure thereby moving the cartridge distally through the compartment distal opening, thereby allowing a user to grip the cartridge distal portion.

8. A drug delivery device as in claim 1, in combination with a drug-filled cartridge, the cartridge comprising an outlet, a generally cylindrical proximal portion and an axially displaceable piston in sliding engagement with the cartridge inner surface, the cartridge comprising an inner circumferential surface proximally of the piston.
